(19) United States
(12) Patent Application Publication
(10) Pub. No.: US 2009/0105843 A1
(43) Pub. Date: Apr. 23, 2009

(54) Method for Bonding a Titanium Based Mesh to a Titanium Based Substrate

(76) Inventor: Kate E. Purnell, Valencia, CA (US)

Correspondence Address: Arthur Freilich;Freilich, Hornbaker & Rosen, 20555 Devonshire Street, # 372, Chatsworth, CA, 91311, US

(21) Appl. No.: 11/990,483

(22) PCT Filed: Aug. 11, 2006
(86) PCT No.: PCT/US2006/031515

§ 371 Date: Feb. 13, 2008

Related U.S. Application Data
(60) Provisional application No. 60/715,217, filed on Sep. 8, 2005.

(51) Int. Cl. A61F 2/02; B23K 20/00

(52) U.S. Cl. 623/23.76; 228/193

(57) ABSTRACT

A method for metallurigically bonding a metal wire mesh to a metal substrate which allows the use of a fragile open weave mesh and/or a thin wall substrate. A thin nickel based layer is placed between a titanium based substrate and a titanium based wire mesh. The mesh and substrate are lightly clamped in intimate contact against the nickel interlayer therebetween, e.g., by wire wrapping. The sandwich, or assembly, (i.e., substrate, interlayer, mesh) is then heated to a temperature, below the melting point of titanium and nickel but sufficient to form a eutectic titanium-nickel alloy (e.g. , Ti₂Ni).

### FIELD OF THE INVENTION

This invention relates generally to metallurgical bonding and more particularly to a method for bonding a porous metal layer, or mesh, e.g., titanium, to a metal substrate, e.g., titanium.

### BACKGROUND

In certain applications, it is desirable to affix a porous metal layer to a metal substrate. For example, certain medical devices employ a biocompatible metal substrate and it is desired to attach a biocompatible metal mesh to the substrate to promote bone and/or tissue ingrowth. International Application PCT/US2004/011079 published 28 Oct. 2004 (incorporated herein by reference) describes one such structure which uses a porous layer attached to the periphery of a percutaneously projecting stud for promoting tissue ingrowth for anchoring the stud and creating an infection resistant barrier,

Although various techniques have been described for bonding a mesh to a substrate, they are generally not suited for applications which use a fragile open weave mesh (e.g., having a pore size on the order of 50 to 200 microns and a porosity between 60 and 95%) and/or a thin substrate wall which can be easily distorted by an applied force. For example, adhesive bonding can be used to affix a mesh to a substrate but the adhesive is typically difficult to control in a blind process and therefore can undesirably fill some of the mesh openings. Moreover, adhesive bonds may be insufficiently strong for some applications and can create biocompatibility and/or tissue reaction problems.

Metallurgical solutions such as laser welding and diffusion bonding generally avoid the limitations of adhesive bonding but introduce other limitations which restrict their use for affixing a fragile open weave mesh to a thin substrate wall. For example, direct laser welding (discussed in U.S. Pat. Nos. 6,049,054 and 5,773,789) is generally not suitable because the low density of the mesh prevents sufficient coalescence of the mesh wires to form an adequate bond. Laser welding with filler material can be used to achieve greater coalescence but the size of the resulting weldment can then obstruct open spaces in the mesh thus reducing the mesh efficacy to promote tissue ingrowth. This is especially true if many such weldments, or tacks, are required.

Diffusion bonding has also been discussed for bonding a mesh pad to a metal substrate. Typically, this involves first diffusion bonding the pad to an underlayer and then bonding the underlayer to the substrate at a lower temperature. The initial diffusion bonding step typically necessitates the use of a high contact pressure for a relatively long time interval. Such a high pressure exerted against a fragile open weave mesh pad can distort and compromise the openness of the mesh and additionally can potentially distort a thin substrate wall. Furthermore, the necessity of applying high pressure and high temperature to nonplanar components (i.e., mesh and substrate) presents a challenging production fixturing problem which can be costly and time consuming.

### SUMMARY OF THE INVENTION

The present invention is directed to a method for metallurgically bonding a metal wire mesh to a metal substrate which method allows the use of a fragile open weave mesh (e.g., having a pore size on the order of 50 to 200 microns and a porosity between 60 and 95%) and/or a thin wall substrate. More particularly, the invention is directed to ametallurgical bonding process which avoids the necessity of applying a pressure sufficiently high to distort the mesh and/or substrate structures and avoids the use of bonding material which potentially could reduce the openness of the mesh.

A preferred bonding process in accordance with the invention will be described with reference to a medical device application which requires affixing an open weave wire mesh structure (e.g., titanium 150×150 mesh twill having a wire diameter of 0.0027″ and a width opening of 100 microns) to a thin housing wall, or substrate, (e.g., titanium having a wall thickness of 0.005″).

In accordance with the invention, a thin nickel based layer is placed between a titanium based substrate and a titanium based wire mesh. The mesh and substrate are lightly clamped in intimate contact against the nickel interlayer therebetween, e.g., by wire wrapping. The sandwich, or assembly, (i.e., substrate, interlayer, mesh) is then heated to a temperature, below the melting point of titanium and nickel but sufficient to form a eutectic titanium-nickel alloy (e.g., Ti₂Ni). For example, in one preferred embodiment, the assembly is processed as follows:
A.) Place assembly in vacuum
B.) Heat to 600° C. in 20 minutes.
C.) Dwell at 600° C. for 10 minutes,
D.) Heat to 1035° C. in 35 minutes,
E.) Dwell at 1035° C. for 10 minutes.
F.) Cool to 600° C. in 5 minutes.
G.) Dwell at 600° C. for 5 minutes
H.) Cool to Ambient Temperature under vacuum in 2 to 3 hours.
I.) Release vacuum.

The foregoing procedure causes the nickel to diffuse into the titanium (mesh and/or substrate) to form a biocompatible alloy extending a short distance beneath the substrate surface. Wherever the nickel is in contact with both the mesh and the substrate, the alloy bonds the mesh wire and substrate together.

If a sufficiently thin layer of nickel is used, all the nickel will be completely absorbed in areas where it contacts the substrate or the mesh, thereby creating a minimal amount of fluid alloy. The nickel interlayer can be introduced either discretely in a sheet of nickel foil, or through conventional processes such as vapor deposition, electroless nickel or electroplated nickel. A 0.0001″ thickness of nickel is suitable to form a metallurgical bond for an exemplary mesh structure as specified above while avoiding excessive alloying with the substrate or filling the mesh openings. A greater nickel thickness, e.g., greater than 0.0002″ can result in excessive fluid alloy formation which can fill the mesh openings and diffuses into the substrate. The appropriate thickness of nickel for other configurations of mesh and substrate thickness can be readily experimentally determined,

### BRIEF DESCRIPTION OF THE FIGURES

FIG. 1 is a perspective exterior view of an exemplary medical device which can be fabricated in accordance with the present invention;

FIG. 2 is an exterior plan view of the medical device of FIG. 1;

FIG. 3 is a sectional view taken substantially along the plane 3-3 of FIG. 2;

FIG. 4 is an exploded perspective view showing the multiple components of the medical device of FIGS. 1-3; and

FIG. 5 is a plot showing the diffusion of nickel into the titanium substrate in accordance with the present invention.

### DETAILED DESCRIPTION

The present invention is directed to a method for bonding a porous metal layer to a metal substrate and to the bonded structure resulting therefrom. Although the invention can be advantageously employed in a variety of applications, it will be described herein primarily with reference to an implantable medical device carrying wire mesh adapted to promote tissue ingrowth.

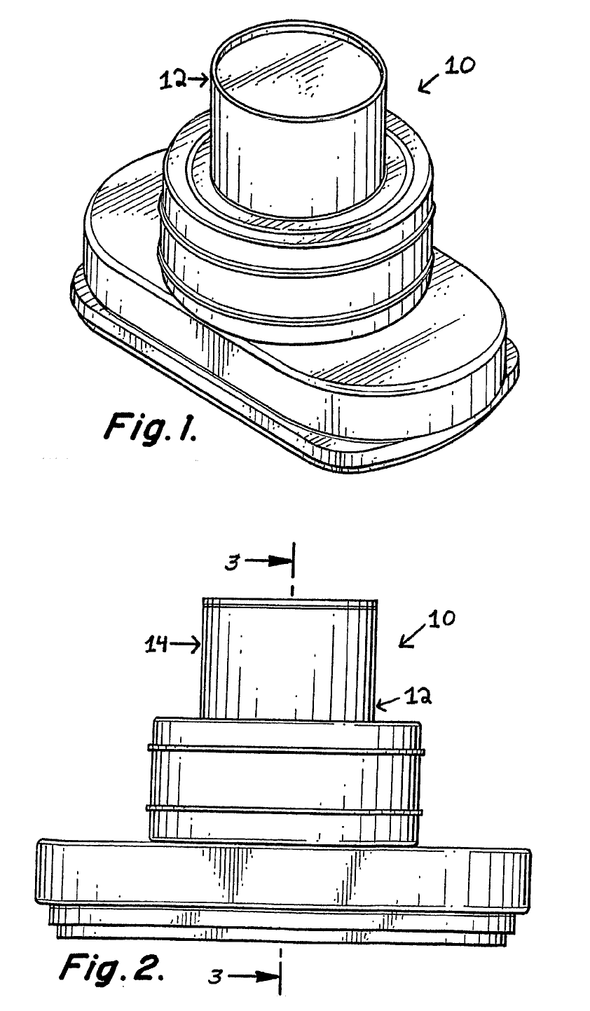

The preferred medical device 10 (as depicted in FIGS. 1-3) is comprised of a housing 12 formed of a biocompatible material, typically titanium. The housing generally comprises a hollow cylindrical stud 14 having an outwardly extending lateral flange 16. The stud 14 is comprised of a thin titanium wall 18 having an outer peripheral surface 20 and an inner peripheral surface 22. The inner peripheral surface 22 surrounds an interior volume 24 intended to accommodate functional components, e.g., a transducer and drive electronics (not shown). The flange 16 defines a lateral shoulder surface 26 which is contiguous with the stud outer peripheral surface 20.

As is discussed in the aforementioned International Application PCT/US2004/011079, it is desirable to affix a porous layer to the stud outer peripheral surface 20 and/or the flange shoulder surface 26 for promoting tissue ingrowth to create an infection resistant barrier and provide effective device anchoring. Although various porous structures can be used, the preferred porous layer which will be assumed herein comprises titanium wire mesh 27 having a pore size on the order of 50 to 200 microns and a porosity of 60 to 95%.

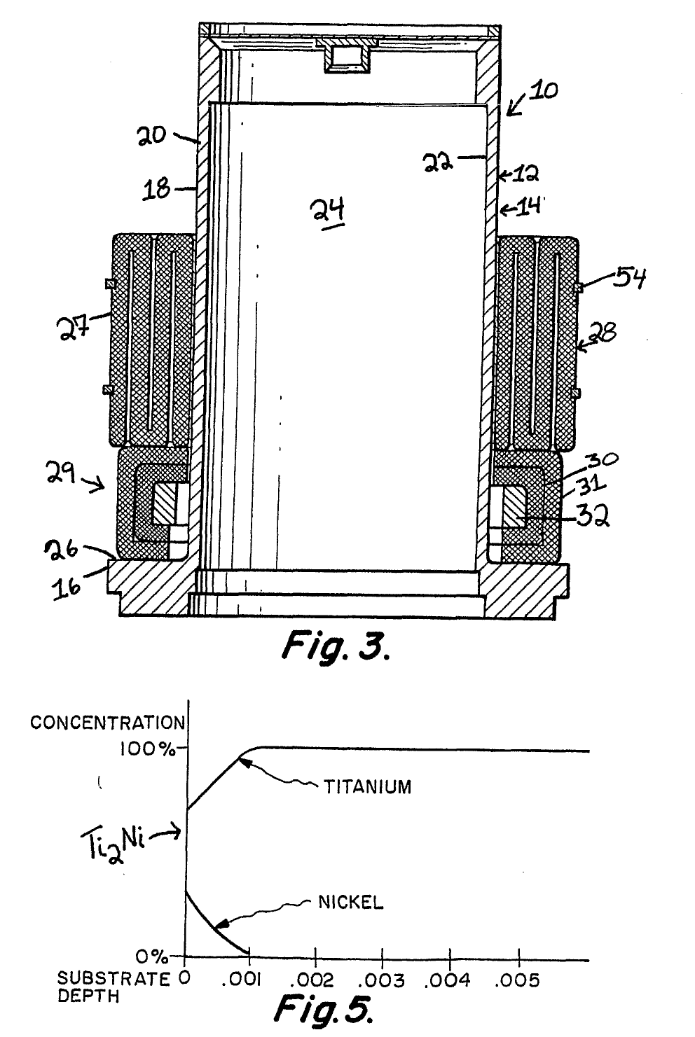

FIG. 3 depicts a stud wire mesh structure 28 formed of folded mesh layers mounted around the stud outer peripheral surface 20 and a second shoulder mesh structure 29 mounted on the shoulder surface 26 and extending around the peripheral surface 20. The mesh structure 29 is comprised of multiple mesh layers 30, 31 supported on a core plate 32 apertured to accommodate the stud 14.

FIG. 4 is an exploded view of the medical device of FIGS. 1-3 and is useful to demonstrate the preferred method in accordance with the invention for bonding wire mesh structures to the surface of housing 12. In accordance with the invention, a thin layer of nickel based material 48, e.g., nickel foil, is placed on the shoulder surface 26 surrounding the stud 14. Then, the shoulder mesh structure 29 (comprised of mesh layers 30, 31 mounted on plate 32) is placed around the stud 14 and on the nickel layer 48. Thereafter, a thin layer of nickel based material 50, e.g., nickel foil, is placed around the stud peripheral surface 20. Subsequently, the stud mesh structure 28 is placed around the nickel layer 50. Light pressure is then applied around the mesh structure 28 (e.g., by wire wraps 54) to assure that the nickel interlayer 50 intimately contacts both the titanium substrate (i.e., stud peripheral surface 20) and the titanium wires of the mesh structure 28. The pressure supplied by wire wraps 54 should be sufficiently light to avoid distorting the mesh structure 28 and/or thin wall substrate 18. Light pressure is also applied (e.g., by wire wraps, not shown) to press mesh structure 29 against shoulder surface 26 to sandwich the nickel interlayer 48 therebetween. It is important for the nickel interlayer 48 to intimately contact both the titanium substrate, i.e., shoulder surface 26, and the mesh structure 29, but it is highly desirable to avoid distorting either the substrate or the mesh structure. Parenthetically, it is also pointed out that FIGS. 3 and 4 also shown a diaphragm or cap 60 which can be secured to the upper end of the housing wall 18 to seal the interior volume 24.

The assembly so formed is then subjected to a heating-cooling procedure to form a biocompatible eutectic alloy of nickel and titanium for bonding the mesh to the substrate. A preferred processing of the assembly fabricated in FIG. 4 comprises the following steps:
A.) Place assembly in vacuum
B.) Heat to 600° C. in 20 minutes.
C.) Dwell at 600° C. for 10 minutes.
D.) Heat to 1035° C. in 35 minutes.
E.) Dwell at 1035° C. for 10 minutes.
F.) Cool to 600° C. in 5 minutes.
G.) Dwell at 600° C. for 5 minutes
H.) Cool to Ambient Temperature under vacuum in 2 to 3 hours.
I.) Release vacuum.

The foregoing procedure causes the nickel to diffuse into the titanium at the eutectic temperature of about 1035° C. to form a biocompatible titanium-nickel alloy (e.g., Ti₂Ni). A bond is formed by the alloy wherever the nickel contacts both titanium substrate and the titanium mesh wires.

If a sufficiently thin nickel interlayer is used, all the nickel will be completely absorbed in areas where it contacts the substrate, the mesh wires, or both, thereby creating a minimal amount of fluid alloy. The nickel interlayer can be introduced either discretely in a sheet of nickel foil, or through conventional processes such as vapor deposition, electroless nickel or electroplated nickel. A 0.0001″ thickness of nickel forms a suitable metallurgical bond for an exemplary mesh structure as specified above while avoiding excessive alloying with the substrate or filling the mesh openings. A greater nickel thickness, e.g., greater than 0.0002″, can result in excessive fluid alloy formation which can fill the mesh openings and diffuses into the substrate. The appropriate thickness of nickel for various configurations of mesh and substrate thickness can be readily experimentally determined.

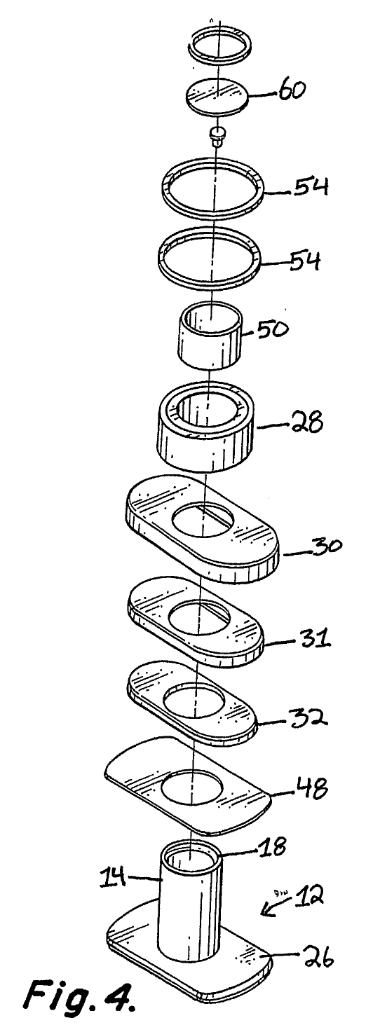

FIG. 5 is a plot depicting the exemplary penetration of nickel into the titanium substrate. At the substrate surface (i.e., zero depth), the eutectic alloy Ti₂Ni can be readily discerned. The concentration of nickel diminishes with depth from about 33% at the substrate surface to about zero at a depth of 0.001 inches. In contrast, the concentration of titanium increases from approximately 66% at the substrate surface to about 100% at a depth of 0.001 inches.

The aforedescribed process is characterized by at least the following attributes. First, the process requires pressure only sufficient to maintain contact between the mesh, nickel interlayer and the substrate. Such light clamping is much simpler to create and maintain, e.g., using wire wrapping, at high temperature than the heavier clamping typically necessary for diffusion bonding. Second, neither the substrate nor the mesh is subjected to deforming pressures, which would be especially problematic for hollow substrates or open-weave meshes subject to elevated temperatures. Third, The entire assembly is subject to a minimal amount of time at high temperature. Fourth, the process requires only a very small amount of nickel to rapidly alloy with the titanium mesh and the substrate at the eutectic temperature indicated (i.e., about 1035° C.). Fifth, the bonding is continuous across the interface of the mesh and substrate, as in diffusion bonding or adhesive bonding, rather than being held at only a discrete number of tack points as in laser welding. Sixth, the interlying layer of nickel is completely absorbed in forming the biocompatible alloy of nickel and titanium thereby avoiding degradation of the mesh porosity. It should be understood that although these multiple attributes are particularly significant when bonding a fragile open weave, or low density, mesh structure to a thin wall substrate, due to the ease of fixturing and processing, this method also provides significant advantages over existing methods of attaching even dense mesh pads to solid implants such as are commonly used in orthopedics.

Although the foregoing describes a particular preferred method for forming a eutectic alloy to bond titanium based wires to a titanium based substrate, it should be understood that variations and modifications may readily occur to those skilled in the art which are nevertheless consistent with the spirit of the invention and within the intended scope of the appended claims.

1. A method of bonding a metal mesh to a metal substrate comprising:
placing a layer of nickel based material on the surface of a titanium based substrate;
placing a titanium based mesh structure on said layer of nickel based material;
forming an assembly by holding said substrate surface and said mesh structure in intimate contact with said layer; and
heating said assembly to a temperature below the melting point of titanium and nickel but sufficient to form a titanium nickel alloy bonding said mesh structure and said substrate.

2. The method of claim 1 wherein said step of heating comprises heating said assembly to a eutectic temperature.

3. The method of claim 2 wherein said step of heating comprises heating said assembly to a temperature of about 1035° C.

4. The method of claim 1 wherein said step of heating comprises heating said assembly in a vacuum to a eutectic temperature of about 1035° C., over a period on the order of 60 minutes and dwelling at said eutectic temperature for a period on the order of 10 minutes.

5. The method of claim 4 further including
cooling said assembly to an ambient temperature while in said vacuum over a period on the order of 2-3 hours.

6. The method of claim 1 wherein said mesh structure is comprised of titanium based wire forming mesh openings on the order of 50 to 200 microns,

7. The method of claim 1 wherein said step of forming said assembly includes holding said substrate and said mesh structure in intimate contact with a force insufficient to significantly distort said mesh structure or substrate.

8. A medical device suitable for implantation in a patient's body, said device comprising:
a substrate defining a titanium bonding surface;
a porous pad comprised of titanium wires; and
a titanium nickel alloy bonding a plurality of said titanium wires to said titanium bonding surface.

9. The device of claim 8 wherein said alloy is diffused into said bonding surface.

10. The device of claim 8 wherein said alloy comprises a eutectic of titanium and nickel.
